(19) United States
(12) Patent Application Publication
(10) Pub. No.: US 2008/0058916 A1
(43) Pub. Date: Mar. 6, 2008

(54) Method of fabricating polymeric self-expandable stent

(76) Inventors: Bin Huang, Pleasanton, CA (US); David C. Gale, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/444,843

(22) Filed: May 31, 2006

(51) Int. Cl. A61F 2/06; B05D 1/12

(52) U.S. Cl. 623/1.2; 427/180; 623/1.54

(57) ABSTRACT

A method of manufacturing a radially expandable an implantable medical device, the method comprising: providing a plurality of fibers, the fibers comprising a polymer; disposing the plurality of fibers on a cylindrical support element to form a tubular structure, the tubular structure having an initial diameter; heat setting the tubular structure such that the temperature of the tubular structure is between Tg to Tm of the polymer while heat setting, wherein the tubular structure is maintained at a heat set diameter which is equal to or substantially equal to the initial diameter; and fabricating an implantable medical device from the tubular structure.

### BACKGROUND OF THE INVENTION

1. Field of the Invention

This invention relates to methods of fabricating implantable medical devices such as stents.

2. Description of the State of the Art

This invention relates to radially expandable endoprostheses which are adapted to be implanted in a bodily lumen. An “endoprosthesis” corresponds to an artificial implantable medical device that is placed inside the body. A “lumen” refers to a cavity of a tubular organ such as a blood vessel. A stent is an example of these endoprostheses. Stents are generally cylindrically shaped devices which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty or valvuloplasty) with apparent success.

Stents have been made of many materials including metals and polymers. Polymer materials include both biostable and biodegradable plastic materials. The cylindrical structure of stents is typically composed of a scaffolding that includes a pattern or network of interconnecting structural elements or struts. The scaffolding can be formed from wires, tubular structures, or planar sheets of material rolled into a cylindrical shape. In addition, a medicated stent may be fabricated by coating the surface of either a metallic or polymeric scaffolding with a polymeric carrier. The polymeric carrier can include an active agent or drug. Furthermore, the pattern that makes up the stent allows the stent to be radially expandable and longitudinally flexible.

A stent treatment involves both delivery and deployment of the stent. “Delivery” refers to introducing and transporting the stent through a bodily lumen to a region requiring treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

It is desirable for a stent to have certain mechanical properties to facilitate delivery and deployment of a stent. For example, longitudinal flexibility is important for successful delivery of the stent. In addition, circumferential strength and rigidity are important for holding open a bodily lumen. The pattern of the stent may be designed to provide longitudinal flexibility.

Some treatments with implantable medical devices require the presence of the device only for a limited period of time. Once treatment is complete, which may include structural tissue support and/or drug delivery, it may be desirable for the stent to be removed or disappear from the treatment location. One way of having a device disappear may be by fabricating at least part of the device from materials that erode or disintegrate when exposed to conditions within the body.

Polymers can be used for making the implantable medical device. A potential shortcoming of implantable medical devices made from polymer material compared to metal stents is that polymer stents typically have less circumferential strength and rigidity. Inadequate circumferential strength potentially contributes to relatively high recoil of polymer devices after implantation into vessels. Furthermore, parts of the device can crack during crimping, especially for brittle polymers. Therefore, methods of manufacturing polymer devices that improve circumferential strength and rigidity are desirable.

### SUMMARY OF THE INVENTION

The invention provides a method of manufacturing a radially expandable an implantable medical device, the method comprising: providing a plurality of fibers, the fibers comprising a polymer; disposing the plurality of fibers on a cylindrical support element to form a tubular structure, the tubular structure having an initial diameter; heat setting the tubular structure such that the temperature of the tubular structure is between Tg to Tm of the polymer while heat setting, wherein the tubular structure is maintained at a heat set diameter which is equal to or substantially equal to the initial diameter; and fabricating an implantable medical device from the tubular structure.

The invention also provides a method of manufacturing an implantable medical device, the method comprising: providing a plurality of fibers, the fibers comprising a polymer; disposing the plurality of fibers on a cylindrical support element to form a tubular structure, the tubular structure having an initial diameter; radially expanding the tubular structure to an expanded diameter such that the expanded diameter is greater than the initial diameter; heat setting the tubular structure such that the temperature of the tubular structure is being at a temperature between Tg to Tm of the polymer while heat setting, wherein the tubular structure is maintained at a heat set diameter equal to or substantially equal to the expanded diameter; and fabricating an implantable medical device from the tubular structure.

### BRIEF DESCRIPTION OF THE DRAWINGS

FIG. 1 depicts an isometric view of a stent that is formed of braided fibers.

FIG. 2 depicts a radial cross section of the stent that is radially expanded.

FIG. 3 depicts a radial cross section of a stent that is crimped and then deployed.

FIG. 4 depicts a tubular structure that is formed on a mandrel, the tubular structure having an initial diameter.

FIG. 5 depicts a tubular structure expanded on a mandrel, the tubular structure having an expanded diameter.

FIG. 6(a) depicts a sliding wedge mandrel before expansion

FIG. 6(b) depicts a sliding wedge mandrel after expansion.

### DETAILED DESCRIPTION OF THE INVENTION

For the purposes of the present invention, the following terms and definitions apply:

“Stress” refers to force per unit area, as in the force acting through a small area within a plane. Stress can be divided into components, normal and parallel to the plane, called normal stress and shear stress, respectively. True stress denotes the stress where force and area are measured at the same time. Conventional stress, as applied to tension and compression tests, is force divided by the original gauge length.

“Strength” refers to the maximum stress along an axis in testing which a material will withstand prior to fracture. The ultimate strength is calculated from the maximum load applied during the test divided by the original cross-sectional area.

“Modulus” may be defined as the ratio of the stress or force per unit area applied to a material divided by the amount of strain resulting from the applied force.

“Strain” refers to the amount of elongation or compression that occurs in a material at a given stress or load.

“Implantable medical device” refers to any type of appliance that is totally or partly introduced into a patient's body, and which is intended to remain there after the procedure. Examples of implantable devices include, without limitation, self-expandable stents, balloon-expandable stents, stent-grafts, vascular grafts, and grafts. An implantable medical device specifically designed and intended solely for the localized delivery of a therapeutic agent is also within the scope of this invention.

The “glass transition temperature,” Tg, is the temperature at which the amorphous domains of a polymer change from a brittle vitreous state to a solid deformable state at atmospheric pressure. In other words, the Tg corresponds to the temperature where the onset of segmental motion in the polymer chains occurs. When an amorphous or semicrystalline polymer is exposed to an increasing temperature, the coefficient of expansion and the heat capacity of the polymer both increase as the temperature is raised, indicating increased molecular motion. As the temperature is raised, the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

“Use” includes manufacturing, assembling (e.g., crimping a stent on a balloon), delivery of a stent through a bodily lumen to a treatment site, and deployment of a stent at a treatment site. For example, a stent requires radial strength and rigidity to resist radial compressive forces. During and after deployment, the stent can be exposed to stress caused by the radial expansion of the stent body. In addition, the scaffolding may be exposed to stress when it is mounted on a catheter from crimping or compression of the stent. These stresses can cause the scaffolding to fracture. Failure of the mechanical integrity of the stent while the stent is localized in a patient can lead to serious risks for a patient.

A “fiber” may be defined as a unit of matter having a length substantially longer than its width or diameter. As used herein, a fiber can include, but is not limited to, a filament, a strip, or a wire.

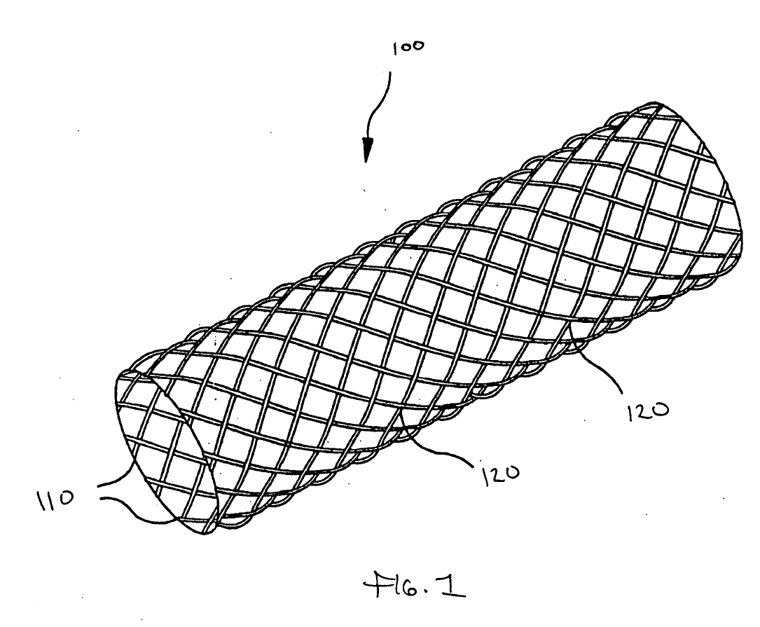

FIG. 1 depicts a side view of a tubular structure of fibers that may have a braided configuration. Tubular structure 100 is formed from two sets of oppositely-directed, parallel, spaced-apart and helically wound fibers 110. The fiber can intersect at points 120 to form an open mesh or weave.

Fibers 110 of the tubular structure form a radially self-expanding body. The fibers are crossed at an axially directed angle and act on one another to create an outwardly directed radial force sufficient to implant the stent in a body vessel upon deployment and to resist inwardly directed forces from the vessel. The body vessel exerts inward radial forces on implanted stents due in part to the cyclical beating of the human heart.

Tubular structure 100 is radially and axially flexible having a predetermined diameter that is variable under axial movement of the ends of the tubular structure relative to each other. Stent 100 includes a plurality of individually rigid but flexible and elastic thread elements or fibers 110, each which extend in a helix configuration along a longitudinal center line of the tubular structure as a common axis. Tubular structure 100 may be formed by a first number of fibers 110 having a common direction of winding but axially displaced relative to each other, and crossing a second number of fibers 110 also axially displaced relative to each other but having an opposite direction of winding.

After being loaded and then released from a deployment device, the stent is radially expanded to a deployment diameter and the fibers cross at an axially directed, smaller angle compared to the angle of the stent before deployment. The deployment diameter of the stent depends on the lumen for the particular application.

To provide strength and rigidity to a stent, the fiber is made to be relatively strong and stiff with a high modulus along the fiber axis. In one embodiment, the polymer of the fiber is selected such that the fiber is at least partially crystalline and exhibits a high modulus. In some embodiments, the fiber for use in fabricating the device has a relatively low crystallinity and high molecular orientation. In one embodiment, the fiber is at least partially crystalline, having a crystallinity of at least 5%, 15%, 30%, or 40% crystallinity.

By drawing the spun fibers, the strength and modulus of the spun fibers for use in fabricating the stent may be increased. Drawing involves applying tension along the fiber axis. Fibers may be drawn while extruded fibers are solidified and/or after they have hardened. Drawing tends to pull the molecular chains of the fiber together and orient the fibers along the fiber axis, forming a considerably stronger and more rigid fiber along the fiber axis. Thus, fibers used to fabricate the stent have high strength and orientation. It is important for the fibers to be able to provide radial strength in the device. Various embodiments of the present invention include a method of manufacturing an implantable medical device, in particular, a self-expanding stent having a braided, radially compressible and heat set structure having greater radial strength.

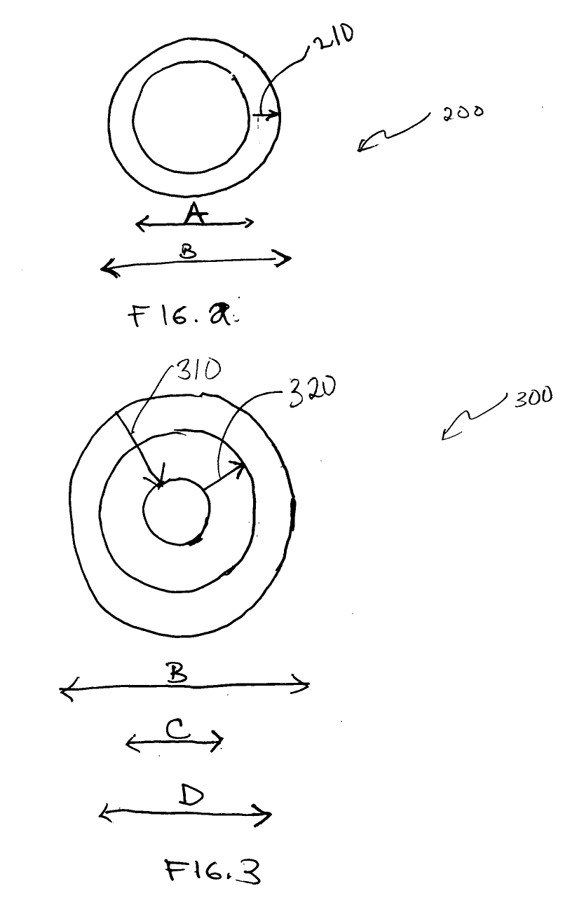

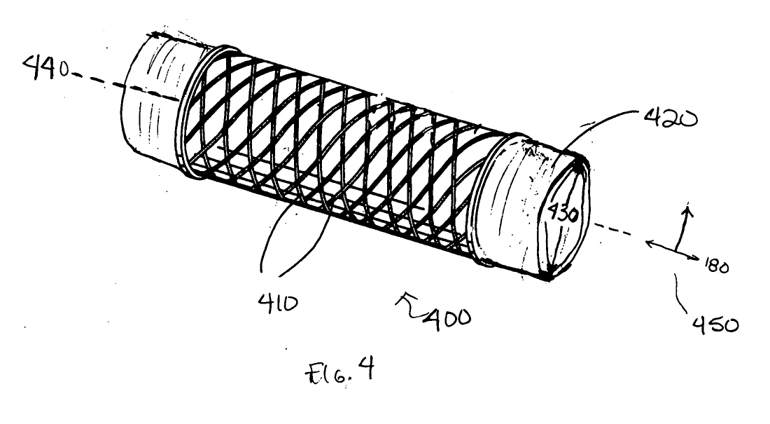

Turning to FIG. 2, depicted is a radial cross section of tubular structure 200 of original diameter (A) and expanded diameter (B). Tubular structure 200 of an original diameter (A), may be heat set at original diameter (A). Alternatively, tubular structure 200 may be radially expanded, as depicted by arrow 210, to expanded diameter (B), and then heat set at radially expanded diameter (B). By radially expanding tubular structure 200, radial strength is imparted in tubular structure 200. Turning now to FIG. 3, depicted is a radial cross section of a tubular structure 300 having an expanded diameter (B), crimped diameter (C) and deployed diameter (D). After heat setting tubular structure of expanded diameter (B), tubular structure 300 may be crimped to crimped diameter (C) as indicated by arrow 310. The crimped stent having crimped diameter (C) can then be deployed from a delivery system, whereby the stent will be “programmed” to self-expand as indicated by arrow 320 to a desired implant or deployed diameter (D). As depicted in FIG. 2 and FIG. 3, the relationship between the diameters of the stent may be B≧A and B>D>C.

As depicted in FIG. 4, embodiments of the present invention include disposing a plurality of fibers 410 on a cylindrical support element, such as a mandrel 420, to form a tubular structure 400, as described above. Tubular structure 400 has an initial diameter 430.

Tubular structure 400 may be heat set such that the temperature of the tubular structure is between Tg to Tm of the polymer. Tubular structure 400 may be maintained at a heat set diameter which is equal to or substantially equal to initial diameter 430 of tubular structure 400. Thus, tubular structure 400 of initial diameter 430 as depicted in FIG. 4 can be heat set at a heat set diameter that is substantially the same diameter as initial diameter 430. By maintaining the diameter of the tubular structure 400 at the same, original diameter 430 as depicted in FIG. 4 during heat setting, the circumferential orientation of the fibers that was created when the tubular structure was formed is maintained.

Fibers 410 may form a woven structure when fibers 410 are disposed onto mandrel 420. A woven structure refers to any structure produced from between one and several hundred or more fibers that are woven, braided, knitted, helically wound, and/or intertwined in any manner, at angles between 0 degrees and 180 degrees with the cylindrical axis of the tubular structure, depending upon the overall geometry and dimensions desired. Fibers 410 may be disposed onto mandrel 420 at various orientations. For example, the fibers may be disposed on mandrel 420 at an angle of from about 120 degrees to about 150 degrees with a cylindrical axis 440 of mandrel 420. Tubular structure 400 has a cylindrical axis 440.

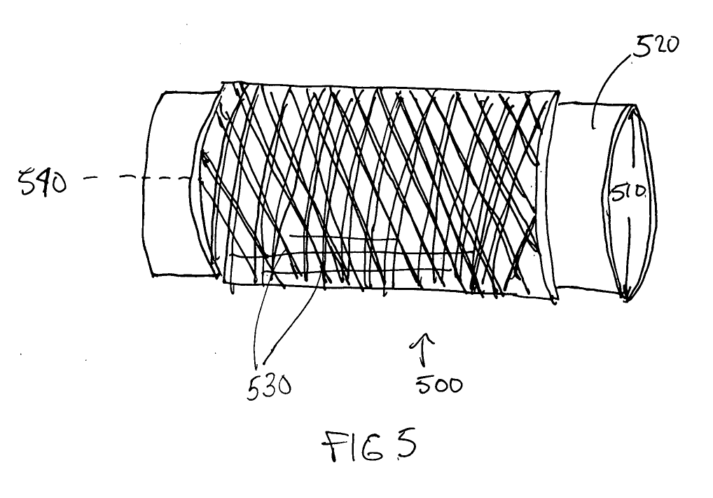

In one embodiment, a method of fabricating a radially expandable endoprosthesis includes radially expanding tubular structure 400 having initial diameter 430, as depicted in FIG. 4, to a radially expanded tubular structure 500 having an expanded diameter 510, as depicted in FIG. 5. Radial expansion of tubular structure 500 causes stretching of fibers 530 along their longitudinal axis. The stretching of fibers induces further alignment of polymer chains along the cylindrical axis 540 of fibers which further increases the radial strength of tubular structure 500.

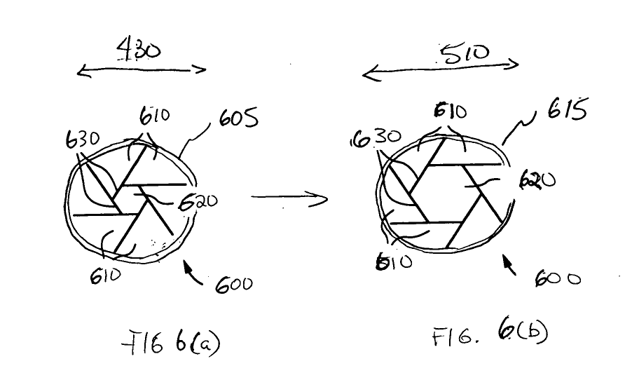

The polymeric tubular structure may be radially expanded from initial diameter 430 of FIG. 4 to expanded diameter 510 of FIG. 5 by various methods. For example, as depicted in FIG. 6(a) and FIG. 6(b), the tubular structure may be radially expanded by a sliding wedge mandrel 600. The polymeric tubular structure is positioned on a mandrel 605 having initial diameter 430 and expanded to expanded diameter 510. As illustrated in FIGS. 6(a) and 6(b), iris mechanism 600 is made up of sliding wedges 610 that form an opening or cavity 620. Iris mechanism 600 has six sliding wedges 610 that form or define opening 620. More wedges may be used to more closely approximate the circular cross-section of a tubular structure. The initial diameter 430 of the tubular structure may be radially expanded to expanded diameter 510 by sliding wedges 610, as illustrated by FIG. 6(b). Walls 630 of wedges 610 move outward as the size of opening 620 increases and acts as restraining surfaces when polymer tubular structure deforms radially in FIG. 6.

Another method that may be used to radially expand the tubular structure to an expanded diameter is by using an inflatable member. An inflatable member is inserted within the tubular structure and inflated, radially expanding the tubular structure. Other means known in the art for radially expanding tubular structure 400 of initial diameter 430, as depicted in FIG. 4, to expanded diameter 510, as depicted in FIG. 5, may also be used in the invention.

Radially expanded tubular structure 500 of FIG. 5 is then heat set at expanded diameter 510 to allow polymer chains to equilibrate to the same configuration (as in FIG. 4) or changed configuration (as in FIG. 5) in response to an elevated temperature. During heat setting, the polymer chains are allowed to adopt an oriented structure at an elevated temperature. Polymer chain alignment is a time and temperature dependent process. Therefore, a period of time may be necessary to allow polymer chains to realign at a given temperature that are stable in a deformed state of a polymeric material. Heat setting may also be facilitated by tension.

The temperature of tubular structure 400 of FIG. 4 or expanded tubular structure 400 of FIG. 4 may be heated for a sufficient period of time to allow polymer chains to equilibrate to the changed configuration to allow the tubular structure to heat set. For example, the selected period of time to heat set the tubular structure can be for about one minute to about two hours, or more narrowly, for about two minutes to about ten minutes. The temperature of the tubular structure while heat setting may be maintained at greater than or equal to Tg of the polymer and less than or equal to Tₘ of the polymer. In one embodiment, the tubular structure is heat set at the temperature greater than temperature of down stream processes in order to minimize temperature effect on crystal structure and final deployment dimension. Further, the temperature that the tubular structure is heated may be to a temperature of about Tg to about Tg+50.

The temperature of the tubular structure may be increased gradually or rapidly while heat setting. Further, the temperature of the tubular structure may be increased and then maintained at a certain temperature. Still further, the temperature of the tubular structure may be increased initially, followed by decreasing the temperature of the tubular structure during the heat setting process. In one embodiment, heat may be applied to the tubular structure while applying inward radial pressure before, after, and/or during the time that the tubular structure is heated.

Thus, expanded tubular structure 500 of expanded diameter 510 as depicted in FIG. 5 can be heat set at a heat set diameter that is substantially the same as expanded diameter 510. By expanding the diameter of the tubular structure 500 as depicted in FIG. 5 to an expanded diameter 510 after the tubular structure is formed, tubular structure 500 increases circumferential orientation of the fibers. Conventional methods of heat setting the formed tubular structure at a smaller diameter than the tubular structure was formed loses circumferential orientation because the tubular structure heat shrinks onto the smaller diameter. Heat setting at a fixed diameter reduces or prevents loss of orientation in fibers.

Tubular structure 400 of FIG. 4 or expanded tubular structure 500 of FIG. 5 may be heat set on the same or a different mandrel on which tubular structure 400 or expanded tubular structure 500 is formed or expanded. For example, tubular structure 400 may be heat set on the same mandrel 420 on which it was formed, or be transferred to a different mandrel to be heat set. Also, radially expanded tubular structure 500 may be heat set on the same mandrel 520 on which it was radially expanded, or radially expanded tubular structure 500 may be heat set on another mandrel to heat set the radially expanded tubular structure 500.

In addition to heat setting, some embodiments may include applying pressure over the tubular structure of fibers before, after, or during heat setting the tubular structure so as to flatten at least some of the fibers and reduce the radial profile of the tubular structure. The tubular structure of fibers may also be heated and flattened in a heated crimper. Heat may be applied to the tubular structure by heating the mandrel. The tubular structure may also be heated by blowing a heated fluid onto the tubular structure such as an inert gas, including argon, nitrogen, etc.

As mentioned previously, after the stent is fabricated, the stent may be crimped to enable delivery of the stent to the treatment site. The stent may be crimped to a smaller diameter (C) than the heat set diameter (FIG. 2).

In general, polymers can be biostable, bioabsorbable, biodegradable, or bioerodable. Biostable refers to polymers that are not biodegradable. Biodegradation of polymers generally refers to changes in physical and chemical properties that occur in a polymer upon exposure to bodily fluids as in a vascular environment. The changes in properties may include a decrease in molecular weight, deterioration of mechanical properties, and decrease in mass due to erosion or absorption. The terms biodegradable, bioabsorbable, and bioerodable, as well as degraded, eroded, and absorbed, are used interchangeably and refer to polymers that are capable of being completely eroded or absorbed when exposed to bodily fluids such as blood and can be gradually resorbed, absorbed, and/or eliminated by the body. In addition, a medicated stent may be fabricated by coating the surface of the stent with an active agent or drug, or a polymeric carrier including an active agent or drug.

The polymeric material for use in fabricating the fiber may be selected from the group consisting of poly-L-lactide, poly-D-lactide, poly(D,L-lactide), poly(ε-caprolactone), polyglycolide, trimethylene carbonate, polydiaxone, and their combinations and copolymers. Other representative examples of polymers that may be used to fabricate the fibers for use in fabricating implantable medical devices disclosed herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

It should also be understood by those skilled on the art that the stent may include an active agent. Alternatively, or in addition, the tubular structure of fibers may first be fabricated into an implantable medical device, such as a stent, and then medicated with an active agent. A medicated stent may be fabricated by coating the surface of the polymeric scaffolding made from the blend with a polymeric carrier that includes a bioactive agent. A bioactive agent can also be incorporated into a polymeric scaffolding made from the blend.

Certain embodiments may include making the tubular structure from at least two types of fibers. In one embodiment, a first fiber may include a first polymer and a second fiber may include a second polymer. The tubular structure may also be made up of composite fibers including the first polymer and the second polymer.

The cross-section of a fiber may be circular, or may be of a different shape such as an oblong shape, for example, an oval or elliptical shape. Fibers with an oblong-shaped cross-section may allow greater surface coverage of a vessel, in addition to providing a smaller radial profile in the stent.

A polymeric fiber may be formed using any of a number of methods known in the art including, but not limited to, melt spinning, wet spinning, dry spinning, gel spinning, electrospinning, or an atomizing process. Fibers may be fabricated with relatively high polymer chain orientation along the fiber axis, and thus relatively high strength and stiffness.

“Spinning” of polymeric fibers generally involves the extrusion or forcing of a thick, viscous fluid, which is either a polymer melt or solution, through the tiny holes of a device called a spinneret to form continuous fibers of a semi-solid polymer.

In melt spinning, the fiber-forming polymer is melted for extrusion through the spinneret and then solidified by cooling. Wet spinning involves forming a fiber from a polymer dissolved in a solvent.

Dry spinning also involves forming fibers from a polymer solution. The polymer solution is pumped through the spinneret. However, instead of precipitating the polymer by dilution or chemical reaction, solidification is achieved by evaporating the solvent in a stream of air or inert gas.

Gel Spinning is a type of wet spinning, but is a special process used to obtain high strength or other special fiber properties. In this process, ultra-high molecular weight polymer is dissolved in a solvent at very low concentration. The concentration is much lower than that typically used in wet spinning and dry spinning processes.

In a dry-jet-wet spinning method, the polymer is not in a true liquid state during extrusion. The polymer chains are bound together at various points in liquid crystal form. The chains are not completely separated, as they would be in a true solution. The method produces strong inter-chain forces in the resulting fibers that can significantly increase the tensile strength of the fibers.

Electrospinning and atomizing processes may be used to produce nanofibers. A “nanofiber” refers to a fiber with a dimension in the range of about 1 nm to about 10,000 nm. Electrospinning makes use of electrostatic and mechanical force to spin fibers from the tip of a fine orifice or spinneret. In electrospinning, a polymer is dissolved in a solvent or a polymer melt and is placed in a spinneret (e.g., a glass pipet) sealed at one end. The spinneret is maintained at positive or negative charge by a power supply, for example.

A stent made from a biodegradable polymer is intended to remain in the body for a duration of time until its intended function of, for example, maintaining vascular patency and/or if the stent is medicated with an active agent or drug, until drug delivery is accomplished. After the process of degradation, erosion, absorption, and/or resorption has been completed, no portion of the biodegradable stent, or a biodegradable portion of the stent remains. In some embodiments, very negligible traces or residue is left behind. The duration can be from about a month to a few years, but is typically in the range of six to eighteen months.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of manufacturing a radially expandable an implantable medical device, the method comprising:
providing a plurality of fibers, the fibers comprising a polymer;
disposing the plurality of fibers on a cylindrical support element to form a tubular structure, the tubular structure having an initial diameter;
heat setting the tubular structure such that the temperature of the tubular structure is between Tg to Tm of the polymer while heat setting, wherein the tubular structure is maintained at a heat set diameter which is equal to or substantially equal to the initial diameter; and
fabricating an implantable medical device from the tubular structure.

2. The method according to claim 1, wherein the implantable medical device is a stent.

3. The method according to claim 1, wherein a temperature of the tubular structure is close to Tm of the polymer while heat setting.

4. The method according to claim 1, wherein providing a plurality of fibers comprises forming fibers by extrusion and drawing.

5. The method according to claim 1, wherein disposing the plurality of fibers comprises helically braiding the fibers on the cylindrical support element.

6. The method according to claim 1, wherein the polymer is selected from the group consisting of poly-L-lactide, poly-D-lactide, polyglycolide, poly(l-lactide-co-glycolide), trimethylene carbonate, polydiaxone, and their combinations and copolymers.

7. The method according to claim 1, wherein the fibers are at least partially crystalline.

8. The method according to claim 1, further comprising crimping the heat set tubular structure to a crimped diameter such that the crimped diameter is less than the heat set diameter.

9. The method according to claim 8, wherein the crimped tubular structure is capable of self-expanding to a diameter greater than the crimped diameter and less than the heat set diameter.

10. The method according to claim 1, wherein the tubular structure is formed on the same cylindrical support element on which the tubular structure is heat set.

11. A method of manufacturing an implantable medical device, the method comprising:
providing a plurality of fibers, the fibers comprising a polymer;
disposing the plurality of fibers on a cylindrical support element to form a tubular structure, the tubular structure having an initial diameter;
radially expanding the tubular structure to an expanded diameter such that the expanded diameter is greater than the initial diameter;
heat setting the tubular structure such that the temperature of the tubular structure is at a temperature between Tg to Tm of the polymer while heat setting, wherein the tubular structure is maintained at a heat set diameter equal to or substantially equal to the expanded diameter; and
fabricating an implantable medical device from the tubular structure.

12. The method according to claim 11, wherein the tubular structure is at a temperature between Tg to Tm of the while radially expanding.

13. The method according to claim 11, wherein the disposed fibers are formed by extrusion and drawing.

14. The method according to claim 11, wherein the implantable medical device is a stent.

15. The method according to claim 11, wherein a temperature of the tubular structure is close to Tm of the polymer while heat setting and/or radial expansion.

16. The method according to claim 11, wherein disposing the plurality of fibers on the cylindrical support element comprises helically braiding the fibers on the cylindrical support element.

17. The method according to claim 11, wherein the polymer is selected from the group consisting of poly-L-lactide, poly-D-lactide, polyglycolide, trimethylene carbonate, polydiaxone, and their combinations and copolymers.

18. The method according to claim 11, wherein the fibers are at least partially crystalline.

19. The method according to claim 11, wherein a sliding wedge mandrel is used to radially expand the tubular structure.

20. The method according to claim 11, wherein expanding the tubular structure comprises using an inflatable member.

21. The method according to claim 11, further comprising crimping the tubular structure to a crimped diameter such that the crimped diameter is less than the heat set diameter.

22. The method according to claim 11, wherein the crimped tubular structure is capable of self-expanding to a diameter greater than the crimped diameter and less than the heat set diameter.

23. The method according to claim 11, wherein the expanded tubular structure is heat set on a cylindrical support element having a different diameter compared to that which the tubular structure was formed.

24. A stent made according to claim 11.
